(19) United States
(12) Patent Application Publication
(10) Pub. No.: US 2022/0268724 A1
(43) Pub. Date: Aug. 25, 2022

(54) PROCESS FOR PRODUCING BANDS FOR BIOMEDICAL SENSORS AND BANDS PRODUCED ACCORDING TO THIS PROCESS

(71) Applicant: LINXENS HOLDING, MANTES LA JOLIE (FR)

(72) Inventors: Daniel Eric, SAINT JEAN LE BLANC (FR); François Germain, PARIS (FR); Simon Vassal, CLICHY (FR)

(21) Appl. No.: 17/625,232

(22) PCT Filed: Jun. 23, 2020
(86) PCT No.: PCT/FR2020/051092

§ 371 Date: Jan. 6, 2022

(30) Foreign Application Priority Data
Jul. 9, 2019 (FR) 19 07663

(51) Int. Cl. G01N 27/327; G01N 33/487

(52) U.S. Cl. CPC G01N 27/3272; G01N 27/3275; G01N 33/48707

(57) ABSTRACT

Process for producing bands for biological measurements on the basis of flexible circuits on a carrier strip, provided with a flexible insulating substrate provided, on at least one of its faces, with conductive tracks, contact pads and electrodes, includes the application, to said face, of masking means leaving the contact pads and/or the electrodes of the band visible and the selective deposition of a layer of noble metal on said contact pads and electrodes through said masking means.

### CROSS-REFERENCE TO RELATED APPLICATIONS

This application is a National Stage of International Application No. PCT/FR2020/051092, having an International Filing Date of 23 Jun. 2020, which designated the United States of America, and which International Application was published under PCT Article 21(2) as WO Publication No. 2021/005279 A1, which claims priority from and the benefit of French Patent Application No. 1907663, filed on 9 Jul. 2019, the disclosures of which are incorporated herein by reference in their entireties.

### BACKGROUND

The present disclosure relates to a process for producing bands for biomedical sensors and bands produced according to this process.

### FIELD

The field of the present disclosure is that of biomedical sensors as bands for biological measurements for diagnostic tests near the client, referred to as point-of-care tests (POCTs), and the present disclosure relates in particular to the measurement of glucose levels for monitoring diabetic patients.

One example of a biosensor band is the glucose meter used by diabetic patients to measure blood glucose concentration. The sensitive element of this biosensor may, in particular, be an enzyme such as glucose oxydase, which converts glucose into gluconic acid which will modify electrical parameters at a band's electrodes on which the biosensor is arranged, these parameters being measured by an associated measurement apparatus to which the band is connected.

### BRIEF DESCRIPTION OF RELATED DEVELOPMENTS

It is known practice to produce bands comprising deposition of carbon by screen printing to produce a biosensor's connection tracks.

It is also known practice to produce measurement bands comprising an insulating flexible substrate on which conductive tracks are produced which comprise a succession of metal layers for which the last layer is a layer of gold and/or palladium.

Such bands, which are generally small in size, comprise, at one end of the tracks, contact pads for connecting the band to contacts of a measurement apparatus and, at a second end of the tracks, electrodes comprising a bioactive material, for example based on an enzyme which forms a biosensor.

The manufacture thereof comprises, for example, the deposition of a layer of resin on a flexible insulating substrate, the deposition of a sheet of copper and then, using a photoresist- and chemical-etching-based technique, the production of copper tracks on which additional metal layers will be deposited using techniques such as physical vapor deposition (PVD), chemical vapor deposition or electrodeposition.

With this technique, the deposits are uniform across all of the tracks, which is expensive for the layer of gold or of palladium.

In addition, there is a process for such bands which comprises the vapor deposition of gold on an insulating substrate followed by laser restructuring of the tracks. This process is expensive and consumes a lot of gold and is not suitable for manufacture using a reel-to-reel process.

### SUMMARY

The present disclosure therefore relates to a process for manufacturing flexible circuits for bands for biological measurements, the aim of which is, in particular, to decrease the amount of noble metal, such as gold or palladium, deposited on conductive tracks of the band by localizing this deposition to the connection regions of the conductive tracks. The process additionally makes it possible to adjust the dimensions of the gold deposit in terms of thickness but also of length and of width.

To that end, the present disclosure proposes a process for producing bands for biological measurements on the basis of flexible circuits on a carrier strip, provided with a flexible insulating substrate provided, on at least one of its faces, with conductive tracks, contact pads and electrodes, said process comprising the application, to said face, of masking means leaving the contact pads and/or the electrodes of the band visible and the selective deposition of a layer of noble metal on said contact pads and/or electrodes through the masking means.

According to one particular embodiment, the production of the carrier strip comprises a succession of steps comprising the deposition or the lamination of a first layer of metal or of a metal alloy, for example a layer of copper or copper alloy, on the carrier strip and production of the tracks, contact pads and electrodes by means of a photolithography or laser-etching process on said layer.

According to one particular embodiment, the process comprises, before selective deposition, deposition of a second layer of metal or of a metal alloy, for example a layer of nickel with or without phosphorus, on the tracks, contact pads and electrodes.

Advantageously, the process is a reel-to-reel process, producing a plurality of bands aligned side by side with one another on the carrier strip.

The reel-to-reel process is advantageously implemented continuously on the carrier strip which is rolled off a first reel and then rolled back onto a second reel.

Preferably, in a later step, the carrier strip is rolled off again and the process comprises deposition of a bioactive material on the electrodes of the measurement bands, and one or more steps of laminating and then cutting out the measurement bands.

According to some embodiments, the masking means for the selective deposition may, in particular, be chosen from a film, an inlay, a tool such as a plastic tool forming a stencil or a masking strip made of foam.

All of these means applied to the tracks of the carrier strip are provided with cut-outs and leave the contact pads and electrodes exposed for the selective metallization of these contact pads and electrodes with the noble metal.

The thickness of the first layer of metal or of a metal alloy may, in particular, be chosen to be between 10 μm and 20 μm.

The deposition of the second layer of metal or of metal alloy may be the deposition of a thickness from 1 μm to 10 μm and preferably from 2 μm to 5 μm.

The selective deposition of the layer of noble metal may advantageously be the deposition of a thickness from 10 nm to 50 nm.

According to one particular embodiment, the strip has a width from 35 mm to 150 mm.

The present disclosure also relates to a band for biological measurements produced according to the process of the present disclosure, in which the material of the flexible insulating substrate is chosen from polyetherimide (PEI) polyethylene terephthalate (PET), polyethylene naphthalate (PEN), polyimide (PI), a glass-epoxy composite and a suitable paper.

According to some embodiments:
the band is such that the tracks are copper tracks with a thickness of copper of between 10 μm and 20 μm,
the tracks are provided with a nickel coating with a thickness of between 2 μm and 5 μm,
the contact pads and the electrodes are covered with noble metal with a deposition thickness from 10 nm to 100 nm.

### BRIEF DESCRIPTION OF THE DRAWINGS

Other features, details and advantages will become apparent from reading the detailed description below and from studying the appended drawings, in which:

FIG. 1 shows an exemplary band applicable to the process of the present application;

FIG. 2 shows a schematic view of steps in an exemplary process according to the present application;

FIG. 3 shows a schematic view of additional process steps.

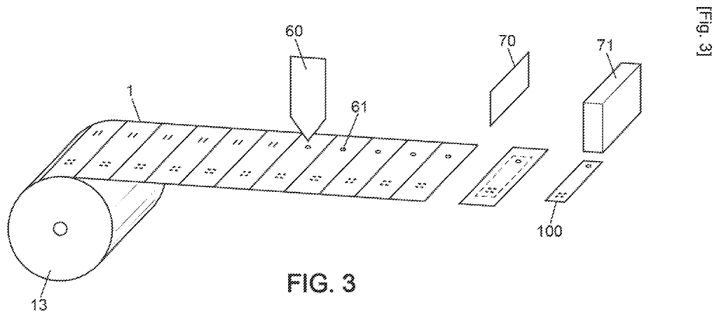

### DETAILED DESCRIPTION

The drawings and description below primarily contain elements of certain character. Therefore, they may not only serve for better understanding of the present disclosure but also contribute to the definition thereof, as the case may be.

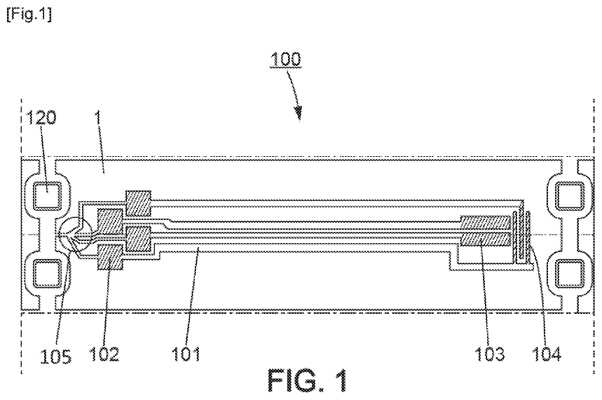

The present disclosure relates to bands for biomedical sensors for taking biological measurements. Such bands, one example of which is schematically shown in FIG. 1 before final cutting-out, comprise a flexible substrate and contact pads 102, here square in shape, which are intended to make an electrical connection with complementary pads of a connection device for the measurement apparatus accommodating the band, and are connected to electrodes 103, 104 by tracks 101. The electrodes accommodate an active product such as an enzyme which, in a reaction with a bodily fluid containing a given marker, for example glucose in the case of a band for monitoring a diabetic person, will modify an electrical characteristic of the band.

The bands are produced on the basis of a carrier strip provided with lateral sprocket holes 120, which forms the flexible insulating substrate for the bands, on which the tracks, pads and electrodes are arranged by means of an, advantageously, reel-to-reel (or roll-to-roll) process.

The material of the carrier strip which will form the flexible insulating substrate for the bands may be chosen from a polyetherimide (PEI), a polyethylene terephthalate (PET), a polyethylene naphthalate (PEN), a polyimide (PI), a glass-epoxy composite or a suitable paper. The width of the strip is adjusted according to the length of the bands which will be produced side by side on the strip. According to the conventional dimensions for bands used in the field and the reel-to-reel devices, the width of the strip may be 35 mm, 70 mm or 150 mm.

The thickness of the carrier strip is in the range from 50 μm to 350 μm.

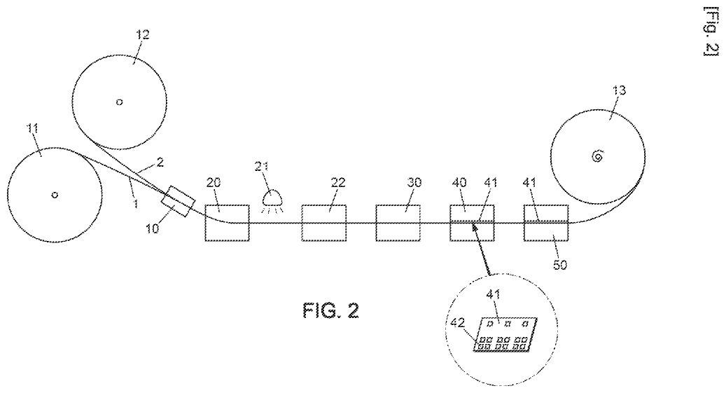

The process schematically shown in FIG. 2 comprises a first step of depositing tracks, pads and electrodes made of metal, for example of copper or of copper-based alloy, on the substrate formed by the carrier strip.

These are, for example, produced by means of photolithography of a copper layer bonded and/or laminated to the substrate or of a layer originating from copper deposition such as electrodeposition. They may also be deposited by lamination.

In the case of bonding or of lamination, the sheet of copper 2 may itself come from a reel 12 which is rolled off and applied continuously to the carrier strip rolled off the reel 11. Bonding is carried out using conventional techniques for producing flexible electronic circuits and, for example, the adhesive may be of two types:

a.—liquid, the gluing process then taking place via coating using a roller or a slot;
b.—in the form of a film, the bonding process then taking place via lamination.

After bonding, a curing step may be carried out at a temperature that may range from 20 to 120° C.

The thickness of the sheet of copper 2 is conventionally chosen to be between 10 μm and 20 μm and more particularly 12 μm or 18 μm.

Next, the process comprises producing copper tracks from the sheet of copper, using a photolithography process which uses a step 20 of putting a photoresist and a mask in place, a step 21 of exposing the unmasked portions of the photoresist to radiation, dissolving the exposed portions of the photoresist followed by a chemical attack 22 which removes the portions of copper from the regions which are no longer covered by the photoresist.

Alternatively, the electrical contact lands may also be mechanically cut out to form a conductive grid which is then roll-bonded to the substrate.

Once the electrical circuit has been made of copper, the process potentially continues with the deposition 30 of a layer of nickel with or without phosphorus on the copper tracks so as to protect them from oxidation.

This layer may be deposited using electrodeposition or autocatalytic deposition.

The layer of nickel has a thickness that affords adequate corrosion resistance, preferably between 2 μm and 5 μm.

Next, the process of the application comprises application 40 of masking means 41 to the track-side of the strip which leave the contact pads and the electrode regions of the band exposed and selective deposition 50 of a layer of noble metal on said contact pads and electrode regions through the masking means 41.

The selective deposition of gold with a thickness from 1 to 100 nanometers, for example performed using electrodeposition or autocatalytic deposition, makes it possible to obtain connection regions of low electrical resistance and with resistance to oxidation.

A number of solutions can be used to produce the masking means. In any case, these means 41 will comprise cut-outs 42 which leave the contact pads and/or electrodes exposed to allow selective deposition of the noble metal thereon.

The masking means may consist of a film provided with openings allowing selective deposition of the noble metal.

They may also consist of an inlay provided with openings allowing selective deposition and applied to the strip as it moves into an electrolytic bath or onto a deposition liquid applicator.

They may additionally consist of a tool, for example a plastic tool forming a stencil, provided with cut-outs leaving the contact pads and electrodes exposed, which is placed on the strip and applied to the tracks of the carrier strip at a station for depositing the noble metal.

They may also consist of a masking strip made of foam applied to the strip and provided with said cut-outs leaving the contact pads and electrodes exposed.

The masking means are positioned in the manufacturing process so as to perform selective deposition through application to the strip as it moves into an electrolytic bath or onto a deposition liquid applicator, the openings leaving the contact pads and/or electrodes exposed. The steps of the process are carried out continuously in succession on the rolled-off carrier strip. The manufacturer producing the carrier strip forming the flexible circuits to produce the bands may then punch the unneeded segments of the tracks 105 and roll the strip with its flexible circuits back onto a reel 13 for potential storage before delivery to the company which performs the step of depositing the biosensor on the bands or for internal transfer to a production line suitable for handling biological products in order to finish the bands.

To finish the bands, they have to be provided with their one or more biosensors. To that end, the carrier strip 1 is rolled off the reel 13 again to carry out the deposition 61 of bioactive material on the electrodes using a deposition device 60. The bioactive material may, for example, be an enzyme suitable for measuring glucose in the treatment of diabetes.

Once this operation has been performed, one or more lamination steps are carried out and the bands 100 are separated in one or more cutting steps 70, 71, for example by means of a first cutter blade which separates the band panels and a punch 71 which trims the bands.

The present disclosure provides an optimized solution for the mass production of biosensor bands, in particular in the context of a reel-to-reel process, and a decrease in the amount of gold or of noble metal required for this manufacture.

What is claimed is:

1. A process for producing bands for biological measurements on the basis of flexible circuits on a carrier strip, provided with a flexible insulating substrate provided, on at least one of its faces, with conductive tracks, contact pads and electrodes, comprising an application, to said face, of masking means leaving the contact pads and/or the electrodes of the band visible and a selective deposition of a layer of noble metal on said contact pads and electrodes through said masking means.

2. The process for producing bands as claimed in claim 1, wherein the production of the carrier strip comprises a succession of steps comprising a deposition or a lamination of a first layer of metal or of a metal alloy on the flexible insulating substrate of said carrier strip and production of the tracks, contact pads and electrodes by means of a photolithography or laser-etching process on said layer.

3. The process as claimed in claim 1, comprising, before said selective deposition, a deposition of a second layer of metal or of a metal alloy on the tracks, contact pads and electrodes.

4. The process as claimed in claim 1, wherein the process is a reel to reel process producing a plurality of bands aligned side by side with one another continuously on the carrier strip which is rolled off a first reel and then rolled back onto a second reel.

5. The process as claimed in claim 4, wherein, the carrier strip being rolled off again, the process comprises a deposition of a bioactive material on the electrodes of the measurement bands, and one or more steps of laminating and then cutting out these measurement bands.

6. The process as claimed in claim 1, wherein the masking means for said selective deposition are chosen from a film, an inlay, a tool such as a plastic tool forming a stencil or a masking strip made of foam and are applied to the carrier strip and provided with cut-outs leaving said contact pads and/or said electrodes exposed for said selective metallization.

7. The process as claimed in claim 2, wherein a thickness of the first layer of metal or of a metal alloy is chosen to be between 10 μm and 20 μm.

8. The process as claimed in claim 3, wherein the deposition (30) of the second layer of metal or of a metal alloy has a thickness from 1 μm to 10 μm and preferably from 2 μm to 5 μm.

9. The process as claimed in claim 1, wherein the selective deposition of the layer of noble metal has a thickness from 10 nm to 100 nm.

10. A band for biological measurements produced according to the process of claim 1, wherein the material of the flexible insulating substrate is chosen from polyetherimide (PEI) polyethylene terephthalate (PET), polyethylene naphthalate (PEN), polyimide (PI), a glass-epoxy composite and a suitable paper.0
